(19) United States
(12) Patent Application Publication
(10) Pub. No.: US 2009/0017007 A1
(43) Pub. Date: Jan. 15, 2009

(54) LIQUID FACTOR VII COMPOSITION

(75) Inventor: Kim Vilbour Andersen, Broenshoej (DK)

Correspondence Address: BAYER HEALTHCARE LLC, Law & Patents, 800 Dwight Way, BERKELEY, CA, 94710, US

(73) Assignee: MAXYGEN HOLDINGS LTD.

(21) Appl. No.: 11/995,866

(22) PCT Filed: Aug. 23, 2006
(86) PCT No.: PCT/DK2006/050036

§ 371 Date: Aug. 25, 2008

Related U.S. Application Data
(60) Provisional application No. 60/711,932, filed on Aug. 26, 2005.

(51) Int. Cl. A61K 38/48; A61P 7/02

(52) U.S. Cl. 424/94.64

(57) ABSTRACT

Storage-stable aqueous pharmaceutical compositions comprising a Factor VII or Factor Vila polypeptide, a buffering agent, and zinc ions (Zn²⁺) as a stabilizer.

### FIELD OF THE INVENTION

The present invention relates to storage-stable aqueous compositions of Factor VII or Factor VIIa comprising zinc ions as a stabilizer.

### BACKGROUND OF THE INVENTION

Factor VII (FVII), an important protein in the blood coagulation cascade, is a vitamin K-dependent plasma protein synthesized in the liver and secreted into the blood as a single-chain glycoprotein with a molecular weight of 53 kDa. The FVII zymogen is converted into an activated form (FVIIa) by proteolytic cleavage at a single site, R152-I153, resulting in two chains linked by a single disulfide bridge. Recombinant human FVIIa is commercially available from Novo Nordisk under the name NovoSeven® and is used for the treatment of bleeding episodes, e.g. in hemophilia or trauma. Recombinantly produced variants of human FVII or FVIIa have also been reported.

NovoSeven® is a freeze-dried FVIIa product which must be reconstituted before use. A vial (1.2 mg) of NovoSeven® contains 1.2 mg recombinant human FVIIa (rhFVIIa), 5.84 mg NaCl, 2.94 mg CaCl₂, 2H₂O, 2.64 mg glycylglycine, 0.14 mg polysorbate 80 and 60.0 mg mannitol; it is reconstituted to pH 5.5 by 2.0 ml water for injection. When reconstituted, the protein is stable for use for 24 hours. No liquid ready-for-use or concentrated FVII products are currently commercially available.

A stable liquid preparation of FVIIa would provide the advantage of easier handling for patients, medical care personnel, etc. Further, in case of emergencies such as severe bleeding caused e.g. by acute trauma or surgery, a ready-to-use soluble formulation of FVIIa would be able to be administered quicker than a dry formulation that must first be reconstituted with water, which could potentially be life-saving and/or prevent complications caused by loss of blood.

Attempts to prevent proteolytic degradation by site-directed mutagenesis at major proteolytic sites are disclosed in WO 88/10295, and attempts to prepare stabilized liquid compositions of FVIIa are disclosed in WO 03/055511, WO 03/055512, WO 2004/082708, WO 2004/112828 and WO 2005/016365.

It is known that zinc ions can function as an in vivo modulator of FVIIa activity. Pedersen et al. (Thromb. Haemost. 65(5): 528-31, 1991) described how the amidolytic and proteolytic activity of FVIIa is inhibited by zinc ions. They found that the amidolytic activity of FVIIa was inhibited by micromolar concentrations of zinc ions in the presence of physiological levels of calcium, with their results suggesting a weak competition between calcium and zinc for a putative zinc binding site on FVIIa. In contrast to the inhibitory effect observed on FVIIa, Pedersen et al. found that zinc ions did not inhibit other vitamin K-dependent proteases tested.

A later study by Petersen et al. (Protein Science 9: 859-866, 2000) similarly found that binding of zinc ions to FVIIa resulted in decreased amidolytic activity and a slightly reduced affinity for tissue factor. The authors suggested that zinc inhibits the activity of FVIIa by specific binding to the calcium binding loop (amino acid residues 210-220).

Bajaj et al. (J. Biol. Chem., Vol. 281 (34):24873-24888, Aug. 25, 2006; originally published online on Jun. 5, 2006) obtained p-aminobenzamidine-VIIa/soluble tissue factor crystals that were used to determine the Ca²⁺, Mg²⁺, Na⁺ and Zn²⁺ sites of FVIIa. They describe that the protease domain has two Zn²⁺ sites unique to FVIIa.

WO 94/22905 discloses a method for the purification of Factor VII in which zinc ions are present in at least one chromatographic purification step in order to purify FVII in its single chain form.

WO 2005/002615 discloses liquid aqueous pharmaceutical compositions comprising a Factor VII polypeptide, a buffer, a non-ionic surfactant, and at least one metal-containing agent selected from the first transition series metals of oxidation state +II, except zinc. According to WO 2005/002615, zinc has properties that are different from the remaining metals of the first transition metal series and is therefore not considered useful in connection with the aqueous FVII compositions described therein.

In spite of the significant clinical advantages that would be provided by a stable liquid formulation of FVIIa, such formulations are not yet available.

### BRIEF DISCLOSURE OF THE INVENTION

The object of the present invention is to provide a stabilized formulation of a Factor VII or VIIa polypeptide, preferably a liquid, aqueous formulation having a sufficient stability to allow storage in liquid form for a desired length of time, without the need for e.g. freezing or freeze-drying, while maintaining a sufficient level of protein activity. This would e.g. make it possible to rapidly administer the polypeptide to a patient without first having to reconstitute a freeze-dried powder in water, thus saving valuable time in acute clinical situations.

One aspect of the invention thus relates to a liquid, aqueous pharmaceutical composition comprising a Factor VII or Factor VIIa polypeptide, a buffering agent, and zinc (Zn²⁺).

Another aspect of the invention relates to a method for producing a storage-stable aqueous composition comprising a Factor VII or Factor VIIa polypeptide, the method comprising mixing the Factor VII or Factor VIIa polypeptide with a buffering agent, zinc (Zn²⁺), and water.

Further aspects of the invention relate to a method of treating or preventing a condition treatable by administration of Factor VIIa, comprising administering to a patient in need thereof a therapeutically effective amount of a composition of the invention, as well as use of a composition of the invention for the manufacture of a medicament for treating or preventing a condition treatable by administration of Factor VIIa.

### DESCRIPTION

### Definitions

In the description and claims below, the follow definitions apply:

The term “FVII” or “FVII polypeptide” refers to a FVII molecule provided in single chain form.

The term “FVIIa” or “FVIIa polypeptide” refers to a FVIIa molecule provided in its activated two-chain form, wherein the peptide bond between R152 and I153 of the single-chain form has been cleaved.

The terms “rFVII” and “rFVIIa” refer to FVII and FVIIa molecules produced by recombinant techniques, respectively. These may have the wild-type human sequence or may be variants of the human sequence.

The terms “hFVII” and “hFVIIa” refer to wild-type human FVII and FVIIa, respectively. The sequence of human FVII/FVIIa is well-known and is disclosed e.g. in U.S. Pat. No. 4,784,950 and in Swiss-Prot under accession number P08709; it is also reproduced below as SEQ ID NO:1.

Unless it is indicated otherwise or apparent from the context, the terms “FVII”, “FVII protein”, “FVII polypeptide” and “Factor VII” as used herein are intended to include both the non-activated and activated forms of FVII, and to include the recombinant wild-type sequence of human FVII as well as variants thereof.

The term “parent” is intended to indicate the FVII or FVIIa molecule from which a FVII or FVIIa variant is derived by way of e.g. substitution, insertion or deletion. Although the parent polypeptide may be any FVII or FVIIa polypeptide, and thus be derived from any origin, e.g. a non-human mammalian origin, the parent polypeptide is normally hFVII or hFVIIa.

A “variant” is a polypeptide which differs in one or more amino acid residues from its parent polypeptide, normally in 1-15 amino acid residues (e.g. in 1, 2, 3, 4, 5, 6, 7, 8, 9, 10, 11, 12, 13, 14 or 15 amino acid residues), such as in 1-10 amino acid residues, e.g. in 1-8, 1-6, 1-5 or 1-3 amino acid residues, where the difference between the parent and the variant is e.g. a substitution, insertion and/or deletion.

The terminology used for identifying amino acid positions is illustrated as follows: G124 indicates that position 124 is occupied by a glycine residue in the amino acid sequence of human FVII. G124R indicates that the glycine residue of position 124 has been substituted with an arginine residue. Alternative substitutions are indicated with a “/”. Multiple substitutions are indicated with a “+”, e.g. K143N+N145S/T means an amino acid sequence which comprises a substitution of the lysine residue in position 143 with an asparagine residue and a substitution of the asparagine residue in position 145 with a serine or a threonine residue. Insertion of an additional amino acid residue, e.g. insertion of an alanine residue after G124, is indicated by G124GA.

### DETAILED DESCRIPTION

According to the present invention, a Factor VII or VIIa polypeptide is formulated in a liquid, aqueous composition containing at least one buffering agent and zinc ions. Preferably, the composition contains the polypeptide in its activated form, i.e. recombinant human Factor VIIa or a variant thereof.

### Zinc

The zinc used in compositions of the invention will normally be in the form of a zinc salt, in particular selected form the group consisting of zinc acetate, zinc bromide, zinc chloride, zinc iodide, zinc fluoride and zinc sulfate. Examples of preferred zinc salts are zinc chloride and zinc acetate.

The zinc salt will generally be present in the compositions in a concentration (calculated as the concentration of the Zn²⁺ ion) of at least about 1 μM, typically at least about 2 μM, such as at least about 5 μM, at least about 10 μM, at least about 20 μM or at least about 50 μM, and up to a maximum of about 100 mM, typically a maximum of about 50 mM, such as a maximum of about 20 mM, a maximum of about 10 mM, a maximum of about 5 mM, a maximum of about 1 mM, a maximum of about 500 μM or a maximum of about 200 μM. The zinc concentration will thus be within the range of 1 μM-100 mM, typically in the range of 1 μM-50 mM, such as 10 μM-10 mM. In one embodiment, the zinc concentration is in the range of 1 μM-1 mM, such as 10-500 μM, e.g. 20-200 μM, for example about 25 μM, about 50 μM, about 100 μM or about 150 μM.

Alternatively, the amount of zinc may be expressed as a molar ratio between zinc (Zn²⁺) and the FVII or FVIIa polypeptide. In this case, the molar ratio will between zinc and the polypeptide will be at least about 0.1, typically at least about 0.2 or at least about 0.5, such as at least about 1, at least about 2 or at least about 5, and up to a maximum of about 1000, typically a maximum of about 500, a maximum of about 200, a maximum of about 150 or a maximum of about 100, such as a maximum of about 50, a maximum of about 25 or a maximum of about 10. Expressed in this manner, the molar ratio between zinc and polypeptide may e.g. be in the range of 0.1-1000, such as 0.2-500, 0.5-200 or 1-100, for example in the range of 1-50, e.g. in the range of 2-25.

pH

The pH of the compositions of the invention is preferably selected so as to minimize pain or discomfort upon administration to patient, and taking into consideration the influence of pH on e.g. stability of the polypeptide and solubility of the zinc salt. However, since FVIIa may be used to treat acute and potentially life-threatening bleeding episodes, e.g. in the case of trauma or intracerebral hemorrhage (ICH), it may in some cases be desirable to formulate the polypeptide using a relatively acidic pH, e.g. down to about 3.0 or perhaps even lower, such as down to about 2.5. The pH will thus generally be in the range of from about 2.5 to about 9.0. Suitable pH ranges may, for example, be from about 2.5 to about 4.0, e.g. about 3.0 or 3.5; from about 4.0 to about 8.0, e.g. from about 5.0 to about 7.0, such as about 5.5, 6.0 or 6.5; or from about 5.0 to about 9.0, e.g. from about 6.0 to about 8.0, such as about 6.5, 7.0 or 7.5.

### Buffering Agents

Suitable buffering agents for use with the present invention include both organic and inorganic acids and salts thereof. Examples of suitable buffering agents include citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffers (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate or maleate buffers; amino acid buffers such as histidine, glycine, glycylglycine, lysine or arginine; trimethylamine salts such as TRIS, as well as MES, PIPES, ACES, BES, TES and HEPES; or imidazole. The buffering agent will generally be present in a concentration of 1-100 mM, such as about 1-50 mM, typically about 1-25 mM, e.g. about 2-20 mM.

### Additional Ingredients

In addition to the polypeptide, the buffering agent and zinc, the compositions of the invention may include one or more additional pharmaceutically acceptable excipients such as a non-ionic surfactant, a tonicity modifying agent, an antioxidant or a preservative.

Non-ionic surfactants or detergents (also known as “wetting agents”) may be present to help solubilize the polypeptide as well as to protect the polypeptide against agitation-induced aggregation, which also reduces the risk of denaturation of the polypeptide upon exposure to shear surface stress. Suitable non-ionic surfactants include, for example, polysorbates (polyoxyethylene sorbitol esters, e.g. polysorbate 20, polysorbate 80, etc.), polyoxamers (polyoxypropylene-polyoxyethylene block copolymers, e.g. poloxamer 184, poloxamer 188, etc.), Pluronic□ polyols, and polyoxyethylene sorbitan monoethers (Tween□-20, Tween□-80, etc.).

Isotonicifiers or tonicity modifying agents are added to ensure isotonicity or to otherwise adjust the tonicity of liquid compositions to a desired level and include polyhydric sugar alcohols, preferably trihydric or higher sugar alcohols, such as glycerin, erythritol, arabitol, xylitol, sorbitol and mannitol, or sugars such as sucrose or trehalose. Also suitable for use as a tonicity modifying agent are neutral salts such as sodium salts, potassium salts, calcium salts or magnesium salts, e.g. with chloride as the counterion. In some cases, it may be advantageous to have a high ionic strength in the composition, e.g. at least 50 mM, at least 100 mM or at least 200 mM, e.g. up to about 500 mM or up to about 1000 mM, for example provided by use of a neutral salt. Such high ionic strength compositions of FVII are described in WO 2004/112828.

Preservatives may be added to retard microbial growth, and are typically added in amounts of e.g. about 0.1%-2% (w/v). Suitable preservatives for use with the present invention include phenol, benzyl alcohol, meta-cresol, ortho-cresol, para-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

In addition, the compositions may comprise miscellaneous excipients such as an antioxidant, for example ascorbic acid, methionine or vitamin E; a bulking agent or filler, e.g. starch; or a chelating agent, e.g. EDTA. The composition may further comprise calcium or magnesium ions, e.g. in the form of calcium chloride or magnesium chloride, for example in a concentration of 1-20 mM, such as about 2-15 mM.

### Stability

The aqueous compositions of the invention are contemplated to be suitable for long-term storage, e.g. in single-use vials that can be stored at a temperature of about 2-8° C. The terms “long-term storage” and “storage-stable” refer to the fact that the aqueous compositions are able to be stored at 2-8° C., such as about 5° C., for an extended period of time, typically a period of at least about 6 months, preferably at least about 1 year, such as up to about 2 years, without suffering any substantial damage to the polypeptide (e.g. proteolysis or other degradation, or aggregation or denaturation, where the degradation, aggregation or denaturation results in a composition that is unsuitable for therapeutic use due to e.g. reduced efficacy or risk of immunogenicity). The storage stability of a polypeptide may e.g. be measured in terms of activity as compared to a reference composition subjected to the same storage conditions, or compared to the same composition that has not been subject to storage or has been subjected to low-temperature storage at e.g. about −80° C. The activity measured may, for example, be amidolytic activity or clotting activity. Suitable assays for determining the amidolytic or clotting activity of FVIIa polypeptides as well as other FVIIa assays are known in the art and are described e.g. in WO 01/58935 and WO 03/093465.

The storage stability may alternatively or additionally be measured in terms of the level of aggregate formation by methods known in the art, for example using size-exclusion chromatography. The improved storage stability of the compositions of the invention is thus intended to comprise physical stability, e.g. reduced aggregate formation, and/or chemical stability, e.g. reduced degradation.

### Freeze-Dried Compositions

Although the compositions of the invention are generally designed for long-term storage in liquid form, it may in some cases be desirable to have a zinc-containing composition according to the invention in lyophilized form, typically in the form of a powder. This could be advantageous e.g. for use under circumstances where maintaining a liquid FVIIa composition at a reduced temperature of about 2-8° C. is difficult, for example for military use where refrigerated storage of a liquid composition may not be possible. The lyophilized composition may e.g. be packaged in single-use vials for reconstitution with water shortly before use. For ease of administration, a freeze-dried zinc-containing FVIIa composition according to the invention may alternatively be packaged in a pre-filled, two-compartment syringe wherein one of the compartments contains the freeze-dried FVIIa composition and the other compartment contains sterile water, thus allowing the dry FVIIa composition to be quickly and easily mixed with water in an appropriate amount immediately prior to administration.

FVII/FVIIa polypeptides

The FVII or FVIIa polypeptides that may be formulated according to the present invention include in particular human recombinant FVII or FVIIa as well as variants thereof, and preferably in the activated form. Factor VII or VIIa variants of interest include, for example, those described in WO 01/58935, WO 03/093465, WO 2004/029091, WO 2004/111242, WO 99/20767, WO 00/66753, WO 88/10295, WO 92/15686, WO 02/29025, WO 01/70763, WO 01/83725, WO 02/02764, WO 02/22776, WO 02/38162, WO 02/077218, WO 03/027147, WO 03/037932, WO 2004/000366, WO 2004/029090, and WO 2004/108763.

The FVII or FVIIa variants may include one or more substitutions, insertions or deletions compared to wild-type human FVII, for example resulting in a variant that differs in 1-15 amino acid residues from the amino acid sequence of wild-type human FVII, typically in 1-10 amino acid residues, e.g. in 1-8 or 1-6 amino acid residues, where the differences in amino acid sequence from the wild-type are typically substitutions. Such substitutions may be performed e.g. with the aim of introducing one or more in vivo glycosylation sites, typically N-glycosylation sites, or introducing one or more PEGylation sites into the protein, and/or for improving or otherwise modifying the clotting activity of the wild-type protein, for example by way of one or more amino acid substitutions in the Gla domain (amino acid residues 1-45 of hFVIIa) to improve clotting activity. Such variants are discussed below and are described in more detail in WO 01/58935, WO 03/093465, WO 2004/029091 and WO 2004/111242. Alternatively, where a FVII or FVIIa variant is designed to function as an anti-coagulant, the composition of the invention may comprise a FVII or FVIIa variant having reduced clotting activity.

Preferred FVII or FVIIa variants suitable for use in the compositions of the invention include at least one modification in the Gla domain (residues 1-45 of human Factor VII) and/or at least one amino acid modification that introduces an attachment site for a non-polypeptide moiety. Non-limiting examples of such modifications are provided in the following.

Modification of the Gla domain: In one embodiment, the FVII or FVIIa variant includes at least one modification in the Gla domain, in particular at least one modification that results in increased phospholipid membrane binding affinity compared to a similar polypeptide without said modification in the Gla domain. Such modifications in the Gla domain are disclosed e.g. in WO 99/20767, WO 00/66753 and WO 03/093465, and include modifications in one or more of positions 10, 11, 28, 32, 33 and 34 relative to SEQ ID NO:1. Preferably, the variant includes modifications in at least position 10 or 32, preferably both. The variant may thus include substitution of a glutamine, a glutamic acid, an aspartic acid or an asparagine residue in position 10, preferably a glutamine residue; and/or substitution of a glutamic acid or an aspartic acid residue in position 32, preferably a glutamic acid. Preferably, the variant includes substitutions at both of positions 10 and 32, more preferably the substitutions P10Q+K32E.

In other embodiments the variant may include substitution of a glutamic acid or a phenylalanine residue at position 28; or substitution of a hydrophobic amino acid residue in position 33, the substitution being selected from the group consisting of D33I, D33L, D33M, D33V, D33F, D33Y and D33W, in particular D33F.

In another embodiment the variant includes substitution of a negatively charged residue in position 34, i.e. A34E or A34D, preferably A34E. Alternatively, the variant may include a hydrophobic amino acid residue introduced by substitution in position 34. In this case, the hydrophobic amino acid residue to be introduced in position 34 may be selected from the group consisting of I, L, M, V, F, Y and W. When position 34 is modified, the substitution A34E will generally be preferred.

In another embodiment the variant includes an amino acid substitution in position 36. Preferably, the amino acid residue to be introduced by substitution in position 36 is a negatively charged amino acid residue, i.e. R36E or R36D, in particular R36E.

In another embodiment the variant includes an amino acid substitution in position 38, in particular a negatively charged amino acid residue introduced by substitution in position 38, i.e. K38E or K38D, in particular K38E.

In another embodiment the variant includes an insertion of at least one (typically one) amino acid residue between position 3 and 4. The inserted amino acid residue is preferably a hydrophobic amino acid residue. Most preferably the insertion is A3AY.

Specific examples of variants having multiple substitutions in the Gla domain include: P10Q+K32E; P10Q+K32E+A34E; P10Q+K32E+R36E; P10Q+K32E+K38E; P10Q+K32E+A34E+R36E; P10Q+K32E+R36E+K38E; P10Q+K32E+A34E+K38E; P10Q+K32E+A34E+R36E+K38E; P10Q+K32E+A34L; P10Q+K32E+A34L+R36E; P10Q+K32E+A34L+K38E; and P10Q+K32E+A34L+R36E+K38E.

Introduction of in vivo glycosylation sites: In another embodiment, the variants used in the compositions of the invention comprise one or more modifications, typically substitutions, that introduce an in vivo N-glycosylation site compared to hFVII with the wild-type sequence. An N-glycosylation site has the sequence N-X-S/T/C, wherein X is any amino acid residue except proline, N is asparagine and S/T/C is either serine, threonine or cysteine, preferably serine or threonine, and most preferably threonine. Attachment sites for in vivo N-glycosylation can therefore be introduced by modification, typically substitution, of one or two amino acid residues in order to obtain the necessary N-X-S/T/C triplet.

Variants having introduced N-glycosylation sites relative to hFVIIa will typically comprise 1-5 additional in vivo N-glycosylation sites, such as 1-4 or 1-3 additional in vivo N-glycosylation sites, e.g. 1, 2 or 3 additional in vivo N-glycosylation sites relative to the native sequence. Human FVII has four naturally occurring glycosylation sites at positions N145, N322, S52 and S60, where S52 and S60 are O-glycosylation sites and N145 and N322 are N-glycosylation sites. Attachment of sugar moieties to one or more in vivo N-glycosylation sites is preferably performed by expression of the polypeptide variant in a host cell capable of in vivo glycosylation.

Specific examples of substitutions creating an in vivo N-glycosylation site include substitutions selected from the group consisting of A51N, G58N, T106N, K109N, G124N, K143N+N145T, A175T, I205S, I205T, V253N, T267N, T267N+S269T, S314N+K316S, S314N+K316T, R315N+V317S, R315N+V317T, K316N+G318S, K316N+G318T, G318N and D334N. Preferred substitutions include one or more of T106N, I205T and V253N.

In one embodiment, only one in vivo N-glycosylation site has been introduced by substitution. In another embodiment, two or more in vivo N-glycosylation sites have been introduced by substitution. Preferred substitutions creating two in vivo N-glycosylation sites include substitutions are selected from the group consisting of T106N+A175T, T106N+I205T, T106N+V253N, T106N+T267N+S269T, A175T+I205T, A175T+V253N, A175T+T267N+S269T, I205T+V253N, I205T+T267N+S269T and V253N+T267N+S269T, more preferably from the group consisting of T106N+I205T, T106N+V253N and I205T+V253N.

In a further embodiment, three or more in vivo N-glycosylation sites have been introduced by substitution. Examples of preferred substitutions creating three in vivo N-glycosylation sites include substitutions selected from the group consisting of I205T+V253N+T267N+S269T and T106N+I205T+V253N.

Variants having a modification in the Gla domain and an introduced N-glycosylation site: In a further embodiment, the compositions of the invention may comprise a FVII or FVIIa variant having at least one modification in the Gla domain and at least one introduced in vivo N-glycosyation site as described in the respective sections above.

Specific examples of “combined” variants having multiple substitutions in the Gla domain and at least one introduced N-glycosylation site include: P10Q+K32E+T106N; P10Q+K32E+A34E+T106N; P10Q+K32E+R36E+T106N; P10Q+K32E+A34E+R36E+T106N; P10Q+K32E+I205T; P10Q+K32E+A34E+I205T; P10Q+K32E+R36E+I205T; P10Q+K32E+A34E+R36E+I205T; P10Q+K32E+V253N; P10Q+K32E+A34E+V253N; P10Q+K32E+R36E+V253N; P10Q+K32E+A34E+R36E+V253N; P10Q+K32E+T106N+I205T; P10Q+K32E+A34E+T106N+I205T; P10Q+K32E+R36E+T106N+I205T; P10Q+K32E+A34E+R36E+T106N+I205T; P10Q+K32E+T106N+V253N; P10Q+K32E+A34E+T106N+V253N; P10Q+K32E+R36E+T106N+V253N; P10Q+K32E+A34E+R36E+T106N+V253N; P10Q+K32E+I205T+V253N; P10Q+K32E+A34E+I205T+V253N; P10Q+K32E+R36E+I205T+V253N; P10Q+K32E+A34E+R36E+I205T+V253N; P10Q+K32E+T106N+I205T+V253N; P10Q+K32E+A34E+T106N+I205T+V253N; P10Q+K32E+R36E+T106N+I205T+V253N; P10Q+K32E+A34E+R36E+T106N+I205T+V253N; P10Q+K32E+A34L+T106N; P10Q+K32E+A34L+I205T; P10Q+K32E+A34L+V253N; P10Q+K32E+A34L+T106N+I205T; P10Q+K32E+A34L+T106N+V253N; P10Q+K32E+A34L+I205T+V253N; P10Q+K32E+A34L+T106N+I205T+V253N; P10Q+K32E+A34L+R36E+T106N; P10Q+K32E+A34L+R36E I205T; P10Q+K32E+A34L+R36E+V253N; P10Q+K32E+A34L+R36E+T106N+I205T; P10Q+K32E+A34L+R36E+T106N+V253N; P10Q+K32E+A34L+R36E+I205T+V253N; and P10Q+K32E+A34L+R36E+T106N+I205T+V253N.

Variants with modifications in the tissue factor binding site: In another embodiment, the compositions of the invention may comprise a FVII or FVIIa variant comprising a substitution in at least one position selected from the group consisting of L39, I42, S43, K62, L65, F71, E82 and F275. These amino acid substitutions in the tissue factor (TF) binding site of the FVII molecule result in an improved clotting activity. Preferred substitutions in these positions in the TF binding site include the following: L39E, L39Q or L39H; 142R; S43Q; K62E or K62R; L65Q or L65S; F71D, F71E, F71N, F71Q or F71Y; E82Q or E82N; F275H. The variant of this embodiment may e.g. comprise one, two or three of these substitutions. Preferred substitutions include one or more of S43Q, K62E, L65Q and F71Y, in particular one or more of S43Q, K62E and L65Q. Further information about variants of this type having modifications in the TF binding site may be found in WO 2004/029091. It will be understood that these substitutions in the TF binding site may if desired be combined with one or more of the other types of modifications described elsewhere herein, e.g. the modifications in the Gla domain as described above, introduction of at least one in vivo N-glycosylation site, and/or conjugation with a PEG polymer as described below.

Variants having an introduced PEGylation site: In a further embodiment, the compositions of the invention may comprise a FVII or FVIIa variant having at least one polymer molecule, in particular a polyethylene glycol (PEG) or other polyalkylene oxide, conjugated to an attachment group selected from the group consisting of a lysine residue, a cysteine residue, an aspartic acid residue, a glutamic acid residue, a histidine residue, and a tyrosine residue, preferably a cysteine or a lysine residue.

Methods for conjugating various polypeptides with a polyethylene glycol moiety (“PEGylation”) are known in the art. For example, WO 01/58935 describes methods by which PEG moieties may be attached to a FVII or FVIIa variant which has been modified relative to hFVII so as to have at least one introduced and/or removed attachment site for PEGylation, for example one or more introduced lysine residues, optionally in combination with removal of one or more lysine residues in positions where PEGylation is not desired, or one or more introduced cysteine residues, in this case optionally in combination with removal of one or more cysteine residues. Additional information regarding PEGylation may be found, e.g., in WO 02/02764, which discloses vitamin K-dependent polypeptides such as FVIIa linked to a PEG polymer, for example wild-type human FVIIa and a variant of FVIIa having the substitutions P10Q and K32E, in WO 96/11953, which describes methods for preparing N-terminally PEGylated proteins, and in the Nektar Advanced PEGylation Catalog 2004, “Polyethylene Glycol and Derivatives for Advanced PEGylation” (Nektar Therapeutics). It will be understood that the FVII or FVIIa variants for use in the compositions of the invention may, in addition to the attachment of one or more PEG polymers, also include one or more of the amino acid modifications otherwise described herein to provide e.g. an increased phospholipid membrane binding affinity and/or an increased tissue factor independent activity, and/or to provide one or more introduced in vivo N-glycosylation sites.

Other modifications: In a further embodiment, variants for use in the compositions of the invention may comprise, in addition to one or more of the modifications described above, at least one further amino acid substitution in a position selected from the group consisting of position 74, 77 and 116, in particular P74S, E77A and/or E116D. In a still further embodiment, the FVII or FVIIa variant may contain mutations known to increase the intrinsic activity of the polypeptide, for example those described in WO 02/22776. For example, the variant may comprise at least one modification in a position selected from the group consisting of 157, 158, 296, 298, 305, 334, 336, 337 and 374. Examples of such substitutions include one or more of V158D, E296D, M298Q, L305V and K337A.

The FVII/FVIIa protein or variant thereof may be produced by any suitable organism, e.g. in mammalian, yeast or bacterial cells, although eukaryotic cells are preferred, more preferably host cells capable of in vivo glycosylation, in particular mammalian cells such as CHO cells, HEK cells or BHK cells. Methods for production of recombinant FVII/FVIIa as well as variants thereof using e.g. mammalian cells are well-known in the art, as are methods for subsequent purification and isolation of the recombinant polypeptides. See, for example, WO 01/58935, WO 03/093465 and WO 2005/002615.

The concentration of the polypeptide in the compositions of the invention may vary and will typically be in the range of about 0.1-5 mg/ml, such as about 0.2-2 mg/ml, e.g. about 0.5-1.5 mg/ml. For example, the concentration may be similar to that of NovoSeven□ after reconstitution with water, which is about 0.6 mg/ml, or slightly higher such as about 1.0 mg/ml. For variants of FVII/FVIIa that have an improved clotting activity compared to rhFVIIa, the concentration of the polypeptide may in some cases be slightly lower, e.g. about 0.1-0.5 mg/ml, such as about 0.2-0.4 mg/ml.

### Additional Aspects

In a further aspect, the invention relates to a method for producing a storage-stable aqueous composition comprising a Factor VII or Factor VIIa polypeptide, the method comprising mixing the Factor VII or Factor VIIa polypeptide with a buffering agent, zinc, and water. It will be understood that the nature and amounts of the buffering agent, zinc, and optional additional components used in this method for stabilizing a FVII/FVIIa composition will be as described above.

In still further aspects, the invention relates to a method of treating or preventing a condition treatable by administration of Factor VIIa, comprising administering to a patient in need thereof a therapeutically effective amount of a composition of the invention as described above, as well as use of a composition of the invention as described above for the manufacture of a medicament for treating or preventing a condition treatable by administration of Factor VIIa. As described above, the composition of the invention will generally be a liquid, aqueous composition, but may in some cases alternatively be in the form of a freeze-dried composition that is reconstituted with water prior to administration.

Conditions treatable by administration of Factor VIIa include blood factor deficiencies, in particular hemophilia A or B, as well as bleeding associated with trauma (both blunt and penetrative trauma), intracerebral hemorrhage (ICH), traumatic brain injury (TBI), burns, variceal bleeds, gastrointestinal bleeding, surgical bleeds, transplantation, fibrinolytic treatment, anticoagulant treatment, postpartum hemorrhage, viral-induced hemorrhage, Von Willebrand disease and thrombocytopenia.

The Factor VII or VIIa compositions of the invention may be administered as single or multiple injections (bolus or transfusion), and may e.g. be administered subcutaneously, intramuscularly or intravenously, but will typically be administered intravenously. The compositions will generally be provided in single-dose form, e.g. in the form of a single-use vial or a pre-filled syringe. The dosage of the polypeptide will e.g. depend on the condition being treated and the weight of the patient, but will often be similar to dosages used for rhFVIIa (NovoSeven□ ), for example from about 20 to about 300 μg/kg, typically from about to about 150 μg/kg, such as from about 40 to about 120 μg/kg

### EXAMPLES

The invention is further described by the following non-limiting examples, which illustrate Factor VIIa compositions according to the invention. In these examples, the zinc ions can e.g. be in the form of zinc chloride or zinc acetate.

### Example 1

| Ingredient | Concentration |
|---|---|
| rhFVIIa or variant | 0.3-1.0 mg/ml, e.g. 0.6 mg/ml (about 12 μM) |
| glycylglycine | 1.0-1.5 mg/ml, e.g. 1.3 mg/ml |
| Zn²⁺ | 50-200 μM, e.g. 100 μM |
| Polysorbate 80 | 0.05-0.2 mg/ml, e.g. 0.1 mg/ml |
| sodium chloride | 2-4 mg/ml, e.g. 3 mg/ml |
| calcium chloride dihydrate | 1-2 mg/ml, e.g. 1.5 mg/ml |
| mannitol | 25-35 mg/ml, e.g. 30 mg/ml |
| pH | 5.5-7.5, e.g. 6.5 |

### Example 2

| Ingredient | Concentration |
|---|---|
| rhFVIIa or variant | 0.8-1.2 mg/ml, e.g. 1.0 mg/ml |
| PIPES | 12-18 mg/ml, e.g. 15 mg/ml |
| Zn²⁺ | 10-100 μM, e.g. 50 μM |
| Poloxamer 188 | 0.5-1.5 mg/ml, e.g. 1.0 mg/ml |
| sodium chloride | 2-4 mg/ml, e.g. 3 mg/ml |
| calcium chloride dihydrate | 1-2 mg/ml, e.g. 1.5 mg/ml |
| methionine | 0.25-0.75 mg/ml, e.g. 0.5 mg/ml |
| sucrose | 60-70 mg/ml, e.g. 65 mg/ml |
| pH | 6.0-7.0, e.g. 6.5 |

### Example 3

| Ingredient | Concentration |
|---|---|
| rhFVIIa or variant | 0.8-1.2 mg/ml, e.g. 1.0 mg/ml |
| PIPES | 12-18 mg/ml, e.g. 15 mg/ml |
| Zn²⁺ | 10-100 μM, e.g. 50 μM |
| Poloxamer 188 | 0.5-1.5 mg/ml, e.g. 1.0 mg/ml |
| sodium chloride | 20-40 mg/ml, e.g. 30 mg/ml |
| calcium chloride dihydrate | 1-2 mg/ml, e.g. 1.5 mg/ml |
| pH | 6.0-7.0, e.g. 6.5 |

### Example 4

| Ingredient | Concentration |
|---|---|
| rhFVIIa or variant | 0.1-0.5 mg/ml, e.g. 0.3 mg/ml |
| glycylglycine | 1.0-1.5 mg/ml, e.g. 1.3 mg/ml |
| Zn²⁺ | 10-100 μM, e.g. 50 μM |
| Poloxamer 188 | 0.5-1.5 mg/ml, e.g. 1.0 mg/ml |
| sodium chloride | 2-4 mg/ml, e.g. 3 mg/ml |
| mannitol | 25-35 mg/ml, e.g. 30 mg/ml |
| pH | 2.5-3.5, e.g. 3.0 |

### Example 5

### Freeze-Dried Composition; Amounts Below are Upon Reconstitution

| Ingredient | Concentration |
|---|---|
| rhFVIIa or variant | 0.3-1.0 mg/ml, e.g. 0.6 mg/ml |
| glycylglycine | 1.0-1.5 mg/ml, e.g. 1.3 mg/ml |
| Zn²⁺ | 20-200 μM, e.g. 100 μM |
| Polysorbate 80 | 0.05-0.2 mg/ml, e.g. 0.1 mg/ml |
| calcium chloride dihydrate | 1-2 mg/ml, e.g. 1.5 mg/ml |
| mannitol | 10-30 mg/ml, e.g. 20 mg/ml |
| sucrose | 30-70 mg/ml e.g. 50 mg/ml |
| pH | 6.5-7.5, e.g. 7.0 |

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

1. A liquid, aqueous pharmaceutical composition comprising a Factor VII or Factor VIIa polypeptide or a Factor VII or Factor VIIa polypeptide variant, a buffering agent, and zinc (Zn²⁺).

2. The composition of claim 1, wherein the Factor VII or Factor VIIa polypeptide is recombinant human Factor VII or human Factor VIIa and wherein the Factor VII or Factor VIIa polypeptide variant comprises an amino acid sequence that differs in no more than 15 amino acid residues from the amino acid sequence of human Factor VIIa.

3. The composition of claim 1, wherein the zinc is in the form of a zinc salt selected form the group consisting of zinc acetate, zinc bromide, zinc chloride, zinc iodide, zinc fluoride and zinc sulfate.

4. The composition of claim 1, comprising zinc in a concentration of 1 μM-1 mM.

5. The composition of claim 1, wherein the molar ratio between zinc and the polypeptide or polypeptide variant is in the range of 0.1-1000.

6. The composition of claim 1, having a pH in the range of from about 2.5 to about 9.0.

7. The composition of claim 1, wherein the buffering agent comprises at least one acid or salt of a component selected from the group consisting of citrate, succinate, tartrate, fumarate, gluconate, oxalate, lactate, acetate, phosphate, maleate histidine, glycine, glycylglycine, lysine, arginine, MES, PIPES, ACES, BES, TES, HEPES, TRIS or imidazole.

8. The composition of claim 1, wherein the buffering agent is present in a concentration of 1-100 mM.

9. The composition of claim 1, wherein the concentration of the polypeptide or polypeptide variant is in the range of 0.1-5 mg/ml.

10. The composition of claim 1, further comprising at least one component selected from a non-ionic surfactant, a tonicity modifying agent, an antioxidant, a preservative, calcium ions and magnesium ions.

11. A method for producing a storage-stable liquid, aqueous composition comprising a Factor VII or Factor VIIa polypeptide or a Factor VII or Factor VIIa polypeptide variant, the method comprising mixing the Factor VII or Factor VIIa polypeptide or the Factor VII or Factor VIIa polypeptide variant with a buffering agent, zinc (Zn²⁺), and water.

12. A method of treating or preventing a condition treatable by administration of a Factor VII or Factor VIIa polypeptide or a Factor VII or Factor VIIa polypeptide variant, comprising administering to a patient in need thereof a therapeutically effective amount of a composition according to claim 1.

13. (canceled)

14. A lyophilized pharmaceutical composition comprising a Factor VII or Factor VIIa polypeptide or a Factor VII or Factor VIIa polypeptide variant, a buffering agent, and zinc (Zn²⁺).

15. A method for producing a lyophilized pharmaceutical composition comprising a Factor VII or Factor VIIa polypeptide or a Factor VII or Factor VIIa polypeptide variant, a buffering agent, and zinc (Zn²⁺), comprising freeze-drying a composition according to claim 1.


SEQUENCE LISTING (1 sequence)

SEQ ID NO: 1 - protein, 406 aa, Homo sapiens [sequence omitted]